(19) United States
(12) Patent Application Publication
(10) Pub. No.: US 2017/0055816 A1
(43) Pub. Date: Mar. 2, 2017

(54) ENDOSCOPE DEVICE

(71) Applicant: OLYMPUS CORPORATION, Tokyo (JP)

(72) Inventor: Jumpei TAKAHASHI, Tokyo (JP)

(73) Assignee: OLYMPUS CORPORATION, Tokyo (JP)

(21) Appl. No.: 15/352,833

(22) Filed: Nov. 16, 2016

Related U.S. Application Data
(63) Continuation of application No. PCT/JP2015/053245, filed on Feb. 5, 2015.

(30) Foreign Application Priority Data
Jun. 16, 2014 (JP) 2014-123403

(51) Int. Cl. A61B 1/06; A61B 1/00; G02B 5/20; G06T 5/20; G06T 3/40; G02B 23/24; A61B 1/04; H04N 5/225

(52) U.S. Cl. CPC A61B 1/0638; A61B 1/04; A61B 1/00009; H04N 5/2256; G06T 5/20; G06T 3/4015; G02B 23/2461; G02B 23/2484; G02B 5/201; H04N 2005/2255; G06T 2207/10024

(57) ABSTRACT

An endoscope device includes: a light source that emits white illumination light including rays of light of red, green, and blue wavelength bands, or emits narrow band illumination light having narrow band light included in each of the blue and green wavelength bands; an image sensor that has pixels arranged in a matrix pattern and performs photoelectric conversion on light received by each pixel to generate an electric signal; and a color filter having filter units arranged on a light receiving surface of the image sensor, each of the filter units being formed of blue filters for transmitting the light of the blue wavelength band, green filters for transmitting the light of the green wavelength band, and red filters for transmitting the light of the red wavelength band, the number of the blue filters and the number of the green filters being larger than the number of the red filters.

### CROSS REFERENCES TO RELATED APPLICATIONS

This application is a continuation of PCT international application Ser. No. PCT/JP2015/053245, filed on Feb. 5, 2015 which designates the United States, incorporated herein by reference, and which claims the benefit of priority from Japanese Patent Application No. 2014-123403, filed on Jun. 16, 2014, incorporated herein by reference.

### BACKGROUND

1. Technical Field

The disclosure relates to an endoscope device configured to be introduced into a living body to obtain an image in the living body.

2. Related Art

Conventionally, endoscope devices have been widely used for various examinations in a medical field and an industrial field. Among them, a medical endoscope device may obtain an image in a body cavity without cutting a subject by inserting an elongated flexible insertion portion on a distal end of which an image sensor having a plurality of pixels is provided, into the body cavity of the subject such as a patient, so that a load on the subject is small and this becomes popular.

As an imaging mode of such endoscope device, white light imaging (WLI) mode in which white illumination light (white illumination light) is used and a narrow band imaging (NBI) mode in which illumination light including two rays of narrow band light included in blue and green wavelength bands (narrow band illumination light) are already well-known in this technical field. Regarding the imaging mode of such endoscope device, it is desirable to observe while switching between the white light imaging mode (WLI mode) and the narrow band imaging mode (NBI mode).

In order to generate a color image to display in the above-described imaging mode, for obtaining a captured image by a single panel image sensor, a color filter obtained by arranging a plurality of filters in a matrix pattern with filter arrangement generally referred to as Bayer arrangement as a unit is provided on a light receiving surface of the image sensor. The Bayer arrangement is such that four filters, each of which transmits light of any of wavelength bands of red (R), green (G), green (G), and blue (B), are arranged in a 2×2 matrix, and G filters which transmit the light of the green wavelength band are diagonally arranged. In this case, each pixel receives the light of the wavelength band transmitted through the filter and the image sensor generates an electric signal of a color component according to the light of the wavelength band.

In the WLI mode, a signal of a green component with which a blood vessel and vasculature of a living body are clearly represented, that is to say, the signal (G signal) obtained by a G pixel (the pixel on which the G filter is arranged; the same applies to an R pixel and a B pixel) contributes to luminance of the image the most. On the other hand, in the NBI mode, a signal of a blue component with which the blood vessel and the vasculature on a living body surface layer are clearly represented, that is to say, the signal (B signal) obtained by the B pixel contributes to the luminance of the image the most.

In the image sensor on which the color filter of the Bayer arrangement is provided, there are two G pixels but there is only one B pixel in a basic pattern. Therefore, in a case of the Bayer arrangement, resolution of the color image obtained in the NBI mode is problematically low.

In order to improve the resolution in the NBI mode, JP 2006-297093 A discloses an image sensor provided with a color filter on which the B pixels are densely arranged as compared to the R pixels and the G pixels.

### SUMMARY

In some embodiments, an endoscope device includes: a light source unit configured to emit white illumination light including rays of light of red, green, and blue wavelength bands, or to emit narrow band illumination light having narrow band light included in each of the blue and green wavelength bands; an image sensor that has a plurality of pixels arranged in a matrix pattern and is configured to perform photoelectric conversion on light received by each of the plurality of pixels to generate an electric signal; a color filter having a plurality of filter units arranged on a light receiving surface of the image sensor, each of the filter units being formed of blue filters for transmitting the light of the blue wavelength band, green filters for transmitting the light of the green wavelength band, and red filters for transmitting the light of the red wavelength band, the number of the blue filters and the number of the green filters being larger than the number of the red filters; a luminance component pixel selecting unit configured to select a luminance component pixel for receiving light of a luminance component, from the plurality of pixels according to types of illumination light emitted by the light source unit; and a demosaicing processing unit configured to generate a color image signal having a plurality of color components based on the luminance component pixel selected by the luminance component pixel selecting unit.

In some embodiments, an endoscope device includes: a light source unit configured to emit white illumination light including rays of light of red, green, and blue wavelength bands, or to emit narrow band illumination light having narrow band light included in each of the blue and green wavelength bands; an image sensor that has a plurality of pixels arranged in a matrix pattern and is configured to perform photoelectric conversion on light received by each of the plurality of pixels to generate an electric signal; a color filter having a plurality of filter units arranged on a light receiving surface of the image sensor, each of the filter units being formed of blue filters for transmitting the light of the blue wavelength band, green filters for transmitting the light of the green wavelength band, and red filters for transmitting the light of the red wavelength band, the number of the blue filters and the number of the green filters being larger than the number of the red filters; a luminance component pixel selecting unit configured to select a luminance component pixel for receiving light of a luminance component, from the plurality of pixels according to types of illumination light emitted by the light source unit; and a motion detection processing unit configured to detect motion of a captured image generated based on the electric signal generated by the pixels in time series, the electric signal being of the luminance component selected by the luminance component pixel selecting unit.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating a configuration of an endoscope device according to an embodiment of the present invention;

FIG. 2 is a schematic diagram illustrating the schematic configuration of the endoscope device according to the embodiment of the present invention;

FIG. 3 is a schematic diagram illustrating a configuration of a pixel according to the embodiment of the present invention;

FIG. 4 is a schematic diagram illustrating an example of a configuration of a color filter according to the embodiment of the present invention;

FIG. 5 is a graph illustrating an example of characteristics of each filter of the color filter according to the embodiment of the present invention, the graph illustrating relationship between a wavelength of light and a transmission of each filter;

FIG. 6 is a graph illustrating relationship between a wavelength and a light amount of illumination light emitted by an illuminating unit of the endoscope device according to the embodiment of the present invention;

FIG. 7 is a graph illustrating relationship between the wavelength and the transmission of the illumination light by a switching filter included in the illuminating unit of the endoscope device according to the embodiment of the present invention;

FIG. 8 is a block diagram illustrating a configuration of a substantial part of a processor of the endoscope device according to the embodiment of the present invention;

FIG. 9 is a schematic view illustrating motion detection between images at different imaging timings performed by a motion vector detection processing unit of the endoscope device according to the embodiment of the present invention;

FIG. 10 is a flowchart illustrating signal processing performed by the processor of the endoscope device according to the embodiment of the present invention;

FIG. 11 is a schematic diagram illustrating a configuration of a color filter according to a first modification of the embodiment of the present invention;

FIG. 12 is a schematic diagram illustrating a configuration of a color filter according to a second modification of the embodiment of the present invention; and

FIG. 13 is a schematic diagram illustrating a configuration of a color filter according to a third modification of the embodiment of the present invention.

### DETAILED DESCRIPTION

Modes for carrying out the present invention (hereinafter, referred to as an “embodiment(s)”) will be hereinafter described. Reference will be made to a medical endoscope device which captures an image in a body cavity of a subject such as a patient to display in the embodiments. The present invention is not limited by the embodiments. The same reference signs are used to designate the same elements throughout the drawings.

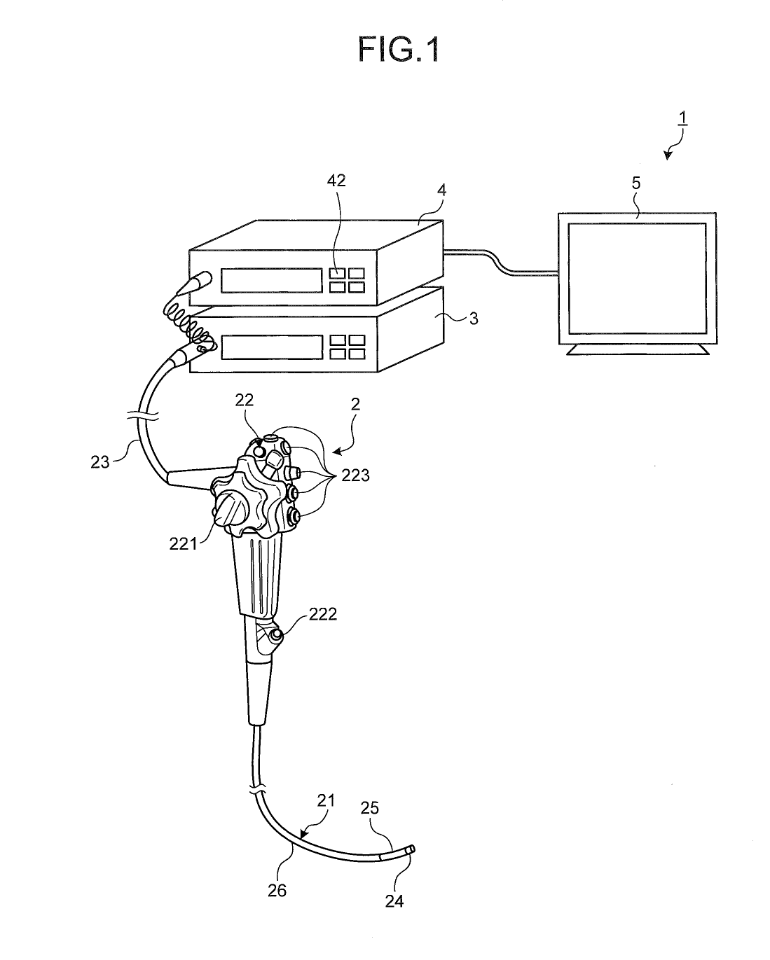

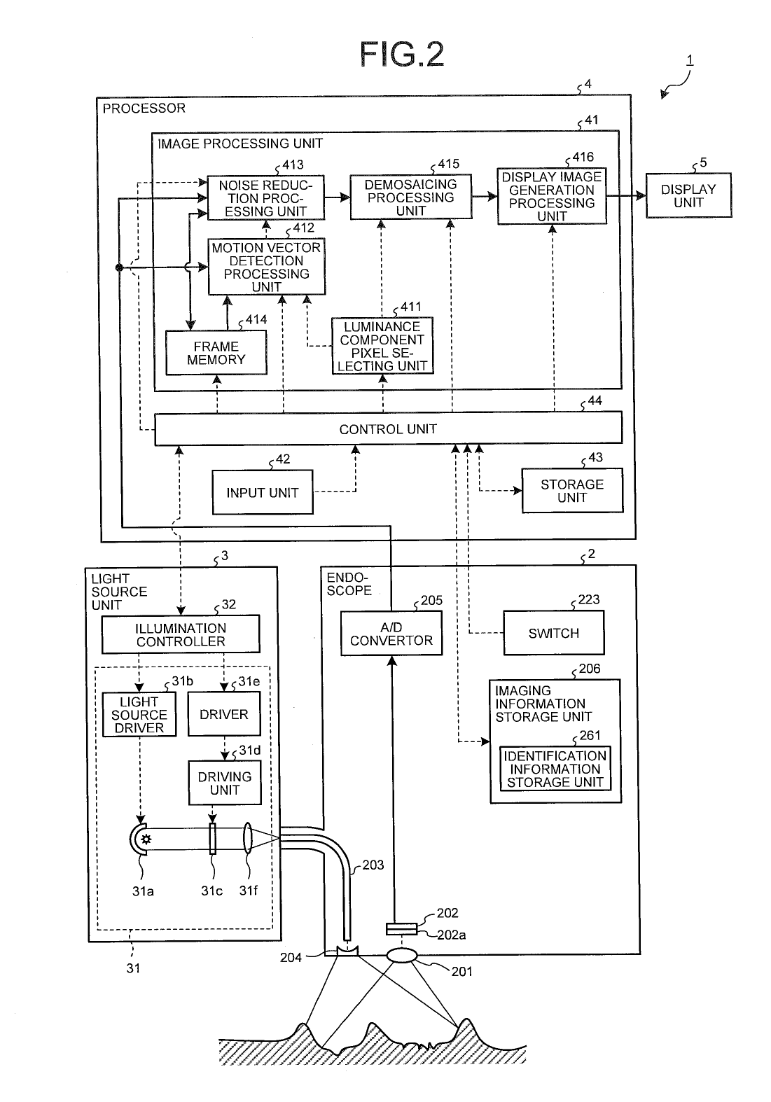

FIG. 1 is a schematic view illustrating a configuration of the endoscope device according to the embodiment of the present invention. FIG. 2 is a schematic diagram illustrating the schematic configuration of the endoscope device according to the embodiment. An endoscope device 1 illustrated in FIGS. 1 and 2 is provided with an endoscope 2 which captures an in-vivo image of an observed region with an insertion portion 21 inserted into the body cavity of the subject, a light source unit 3 which generates illumination light emitted from a distal end of the endoscope 2, a processor 4 which performs predetermined image processing on an electric signal obtained by the endoscope 2 and generally controls operation of an entire endoscope device 1, and a display unit 5 which displays the in-vivo image on which the processor 4 performs the image processing. The endoscope device 1 obtains the in-vivo image in the body cavity with the insertion portion 21 inserted into the body cavity of the subject such as the patient. A user such as a doctor observes the obtained in-vivo image to examine whether there is a bleeding site or a tumor site being sites to be detected. In FIG. 2, a solid line arrow indicates transmission of the electric signal regarding the image and a broken line arrow indicates transmission of the electric signal regarding control.

The endoscope 2 is provided with the insertion portion 21 in an elongated shape having flexibility, an operating unit 22 connected to a proximal end side of the insertion portion 21 which accepts an input of various operation signals, and a universal code 23 extending in a direction different from a direction in which the insertion portion 21 extends from the operating unit 22 including various cables connected to the light source unit 3 and the processor 4 embedded therein.

The insertion portion 21 includes a distal end portion 24 in which an image sensor 202 including pixels (photo diodes) which receive light arranged in a matrix pattern which generates an image signal by performing photoelectric conversion on the light received by the pixels is embedded, a bendable portion 25 formed of a plurality of bending pieces, and an elongated flexible tube portion 26 with flexibility connected to a proximal end side of the bendable portion 25.

The operating unit 22 includes a bending nob 221 which bends the bendable portion 25 in up-and-down and right-and-left directions, a treatment tool insertion unit 222 through which treatment tools such as in-vivo forceps, an electric scalpel, and an examination probe are inserted into the body cavity of the subject, and a plurality of switches 223 which inputs an instruction signal for allowing the light source unit 3 to perform illumination light switching operation, an operation instruction signal of the treatment tool and an external device connected to the processor 4, a water delivery instruction signal for delivering water, a suction instruction signal for performing suction and the like. The treatment tool inserted through the treatment tool insertion unit 222 is exposed from an aperture (not illustrated) through a treatment tool channel (not illustrated) provided on a distal end of the distal end portion 24. The switch 223 may also include an illumination light changeover switch for switching the illumination light (imaging mode) of the light source unit 3.

The universal code 23 at least includes a light guide 203 and a cable assembly formed of one or more signal lines embedded therein. The cable assembly being the signal line which transmits and receives the signal between the endoscope 2 and the light source unit 3 and processor 4 includes the signal line for transmitting and receiving setting data, the signal line for transmitting and receiving the image signal, the signal line for transmitting and receiving a driving timing signal for driving the image sensor 202 and the like.

The endoscope 2 is provided with an imaging optical system 201, the image sensor 202, the light guide 203, an illumination lens 204, an A/D converter 205, and an imaging information storage unit 206.

The imaging optical system 201 provided on the distal end portion 24 collects at least the light from the observed region. The imaging optical system 201 is formed of one or a plurality of lenses. The imaging optical system 201 may also be provided with an optical zooming mechanism which changes an angle of view and a focusing mechanism which changes a focal point.

The image sensor 202 provided so as to be perpendicular to an optical axis of the imaging optical system 201 performs the photoelectric conversion on an image of the light formed by the imaging optical system 201 to generate the electric signal (image signal). The image sensor 202 is realized by using a charge coupled device (CCD) image sensor, a complementary metal oxide semiconductor (CMOS) image sensor and the like.

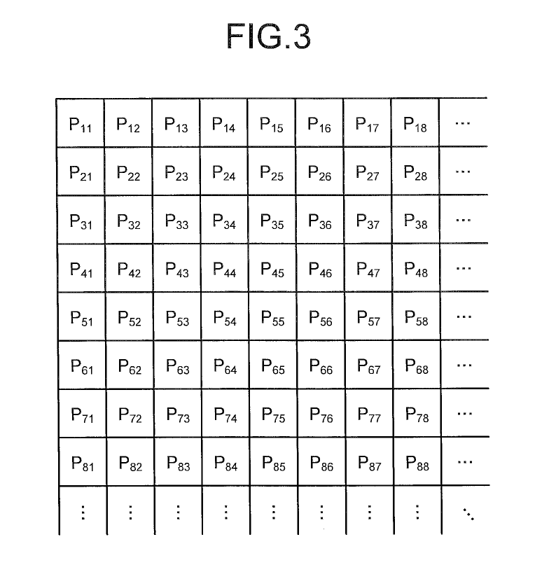

FIG. 3 is a schematic diagram illustrating a configuration of pixels of the image sensor according to the embodiment. The image sensor 202 includes a plurality of pixels which receives the light from the imaging optical system 201 arranged in a matrix pattern. The image sensor 202 generates an imaging signal made of the electric signal generated by the photoelectric conversion performed on the light received by each pixel. The imaging signal includes a pixel value (luminance value) of each pixel, positional information of the pixel and the like. In FIG. 3, the pixel arranged in ith row and jth column is denoted by a pixel Pᵢⱼ.

The image sensor 202 is provided with a color filter 202a including a plurality of filters, each of which transmits light of an individually set wavelength band arranged between the imaging optical system 201 and the image sensor 202. The color filter 202a is provided on a light receiving surface of the image sensor 202.

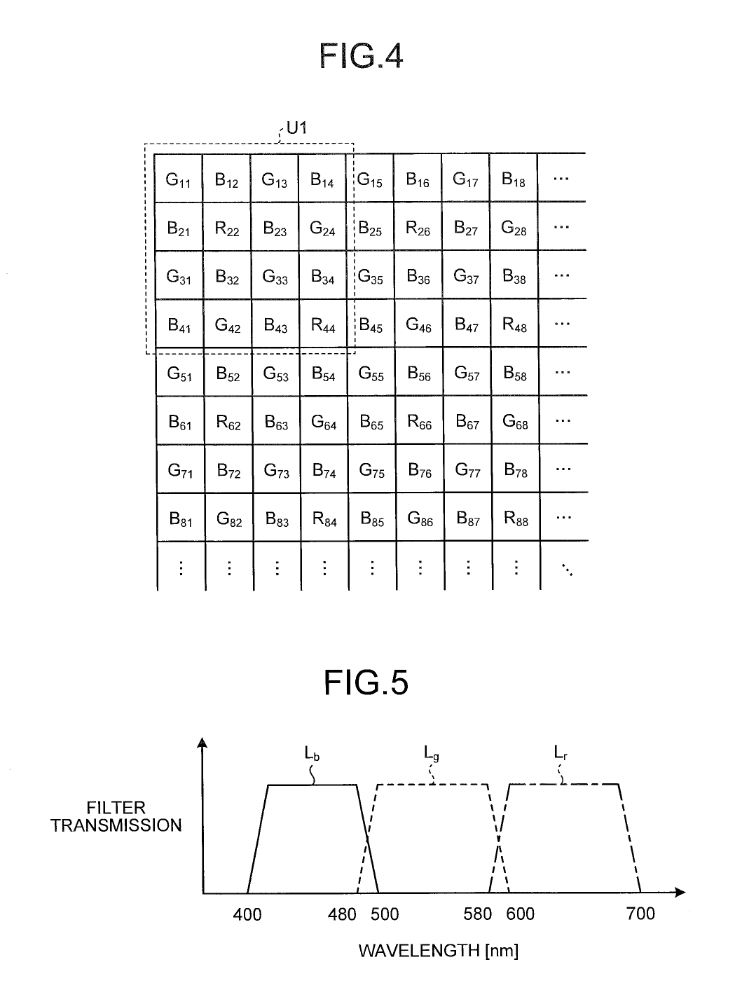

FIG. 4 is a schematic diagram illustrating an example of a configuration of the color filter according to the embodiment. The color filter 202a according to the embodiment is obtained by arranging filter units U1, each of which is formed of 16 filters arranged in a 4×4 matrix, in a matrix pattern according to arrangement of the pixels Pᵢⱼ. In other words, the color filter 202a is obtained by repeatedly arranging filter arrangement of the filter unit U1 as a basic pattern. One filter which transmits the light of a predetermined wavelength band is arranged on a light receiving surface of each pixel. Therefore, the pixel Pᵢⱼ on which the filter is provided receives the light of the wavelength band which the filter transmits. For example, the pixel Pᵢⱼ on which the filter which transmits the light of a green wavelength band is provided receives the light of the green wavelength band. Hereinafter, the pixel Pᵢⱼ which receives the light of the green wavelength band is referred to as a G pixel. Similarly, the pixel which receives the light of a blue wavelength band is referred to as a B pixel, and the pixel which receives the light of a red wavelength band is referred to as an R pixel.

Herein, the filter unit U1 transmits the light of a blue (B) wavelength band H_{B}, a green (G) wavelength band H_{G}, and a red (R) wavelength band H_{R}. In addition, the filter unit U1 is formed of one or a plurality of blue filters (B filters) which transmits the light of the wavelength band H_{B}, green filters (G filters) which transmits the light of the wavelength band H_{G}, and red filters (R filters) which transmits the light of the wavelength band H_{R}; the numbers of the B filters and the G filters are selected to be larger than the number of the R filters. The blue wavelength band H_{B} is 400 nm to 500 nm, the green wavelength band H_{G} is 480 nm to 600 nm, and the red wavelength band H_{R} is 580 nm to 700 nm, for example.

As illustrated in FIG. 4, the filter unit U1 according to the embodiment is formed of eight B filters which transmit the light of the wavelength band H_{B}, six G filters which transmit the light of the wavelength band H_{G}, and two R filters which transmit the light of the wavelength band H_{R}. In the filter unit U1, the filters which transmit the light of the wavelength band of the same color (same color filters) are arranged so as not to be adjacent to each other in a row direction and a column direction. Hereinafter, when the B filter is provided at a position corresponding to the pixel Pᵢⱼ, the B filter is denoted by Bᵢⱼ. Similarly, when the G filter is provided at a position corresponding to the pixel Pᵢⱼ, the G filter is denoted by Gᵢⱼ, and when the R filter is provided there, the R filter is denoted by Rᵢⱼ.

The filter unit U1 is configured such that the numbers of the B filters and the G filters are not smaller than one third of the total number of the filters (16 filters) constituting the filter unit U1, and the number of the R filters is smaller than one third of the total number of the filters. In the color filter 202a (filter unit U1), a plurality of B filters is arranged in a checkerboard pattern.

FIG. 5 is a graph illustrating an example of characteristics of each filter of the color filter according to the embodiment, the graph illustrating relationship between a wavelength of the light and a transmission of each filter. In FIG. 5, a transmission curve is normalized such that maximum values of the transmission of respective filters are the same. A curve L_{b} (solid line), a curve L_{g} (broken line), and a curve Lᵣ (dash-dotted line) in FIG. 5 indicate the transmission curves of the B filter, G filter, and R filter, respectively. As illustrated in FIG. 5, the B filter transmits the light of the wavelength band H_{B}. The G filter transmits the light of the wavelength band H_{G}. The R filter transmits the light of the wavelength band H_{R}.

Returning to the description of FIGS. 1 and 2, the light guide 203 formed of a glass fiber and the like serves as a light guide path of the light emitted by the light source unit 3.

The illumination lens 204 provided on a distal end of the light guide 203 diffuses the light guided by the light guide 203 to emit out of the distal end portion 24.

The A/D converter 205 A/D converts the imaging signal generated by the image sensor 202 and outputs the converted imaging signal to the processor 4.

The imaging information storage unit 206 stores data including various programs for operating the endoscope 2, various parameters required for the operation of the endoscope 2, identification information of the endoscope 2 and the like. The imaging information storage unit 206 includes an identification information storage unit 261 which stores identification information. The identification information includes specific information (ID), a model year, specification information, and a transmission system of the endoscope 2, arrangement information of the filters regarding the color filter 202a and the like. The imaging information storage unit 206 is realized by a flash memory and the like.

Next, a configuration of the light source unit 3 is described. The light source unit 3 is provided with an illuminating unit 31 and an illumination controller 32.

The illuminating unit 31 switches between a plurality of rays of illumination light of different wavelength bands to emit under the control of the illumination controller 32. The illuminating unit 31 includes a light source 31a, a light source driver 31b, a switching filter 31c, a driving unit 31d, a driver 31e, and a condenser lens 31f.

The light source 31a emits white illumination light including rays of light of the red, green, and blue wavelength bands H_{B}, H_{G}, and H_{R} under the control of the illumination controller 32. The white illumination light generated by the light source 31a is emitted outside from the distal end portion 24 through the switching filter 31c, the condenser lens 31f, and the light guide 203. The light source 31a is realized by using a light source which generates white light such as a white LED and a xenon lamp.

The light source driver 31b supplies the light source 31a with current to allow the light source 31a to emit the white illumination light under the control of the illumination controller 32.

The switching filter 31c transmits only blue narrow band light and green narrow band light out of the white illumination light emitted by the light source 31a. The switching filter 31c is removably arranged on an optical path of the white illumination light emitted by the light source 31a under the control of the illumination controller 32. The switching filter 31c is arranged on the optical path of the white illumination light to transmit only the two rays of narrow band light. Specifically, the switching filter 31c transmits narrow band illumination light including light of a narrow band T_{B} (for example, 400 nm to 445 nm) included in the wavelength band H_{B} and light of a narrow band T_{G} (for example, 530 nm to 550 nm) included in the wavelength band H_{G}. The narrow bands T_{B} and T_{G} are the wavelength bands of blue light and green light easily absorbed by hemoglobin in blood. It is sufficient that the narrow band T_{B} at least includes 405 nm to 425 nm. Light limited to this band to be emitted is referred to as the narrow band illumination light and observation of the image by using the narrow band illumination light is referred to as a narrow band imaging (NBI) mode.

The driving unit 31d formed of a stepping motor, a DC motor and the like puts or removes the switching filter 31c on or from the optical path of the light source 31a.

The driver 31e supplies the driving unit 31d with predetermined current under the control of the illumination controller 32.

The condenser lens 31f collects the white illumination light emitted by the light source 31a or the narrow band illumination light transmitted through the switching filter 31c, and outputs the white illumination light or the narrow band illumination light outside the light source unit 3 (light guide 203).

The illumination controller 32 controls the type (band) of the illumination light emitted by the illuminating unit 31 by controlling the light source driver 31b to turn on/off the light source 31a and controlling the driver 31e to put or remove the switching filter 31c on/from the optical path of the light source 31a.

Specifically, the illumination controller 32 controls to switch the illumination light emitted from the illuminating unit 31 to the white illumination light or the narrow band illumination light by putting or removing the switching filter 31c on or from the optical path of the light source 31a. In other words, the illumination controller 32 controls to switch between a white light imaging (WLI) mode in which the white illumination light including rays of light of the wavelength bands H_{B}, H_{G}, and H_{R} is used and the narrow band imaging (NBI) mode in which the narrow band illumination light including rays of light of the narrow bands T_{B} and T_{G} is used.

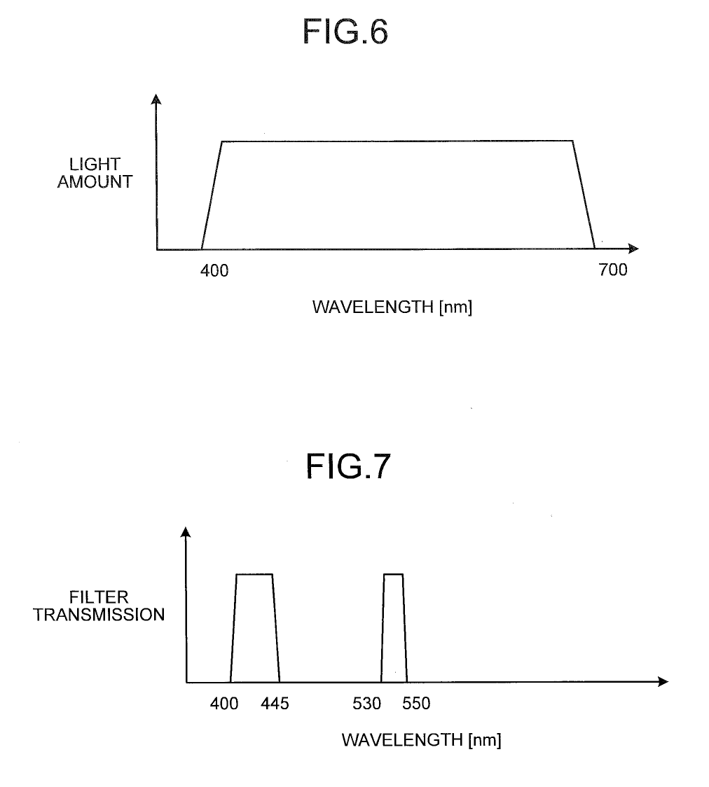

FIG. 6 is a graph illustrating relationship between the wavelength and a light amount of the illumination light emitted by the illuminating unit of the endoscope device according to the embodiment. FIG. 7 is a graph illustrating relationship between the wavelength of the illumination light and the transmission by the switching filter included in the illuminating unit of the endoscope device according to the embodiment. When the switching filter 31c is removed from the optical path of the light source 31a by the control of the illumination controller 32, the illuminating unit 31 emits the white illumination light including rays of light of the wavelength bands H_{B}, H_{G}, and H_{R} (refer to FIG. 6). On the other hand, when the switching filter 31c is put on the optical path of the light source 31a by the control of the illumination controller 32, the illuminating unit 31 emits the narrow band illumination light including rays of light of the narrow bands T_{B} and T_{G} (refer to FIG. 7).

Next, a configuration of the processor 4 is described. The processor 4 is provided with an image processing unit 41, an input unit 42, a storage unit 43, and a control unit 44.

The image processing unit 41 executes predetermined image processing based on the imaging signal from the endoscope 2 (A/D converter 205) to generate a display image signal for the display unit 5 to display. The image processing unit 41 includes a luminance component pixel selecting unit 411, a motion vector detection processing unit 412 (motion detection processing unit), a noise reduction processing unit 413, a frame memory 414, a demosaicing processing unit 415, and a display image generation processing unit 416.

The luminance component pixel selecting unit 411 determines the illumination light switching operation by the illumination controller 32, that is to say, determines which of the white illumination light and the narrow band illumination light the illumination light emitted by the illuminating unit 31 is. The luminance component pixel selecting unit 411 selects a luminance component pixel (pixel which receives light of a luminance component) used by the motion vector detection processing unit 412 and the demosaicing processing unit 415 according to the determined illumination light.

The motion vector detection processing unit 412 detects motion of the image as a motion vector by using a pre-synchronization image according to the imaging signal from the endoscope 2 (A/D converter 205) and a pre-synchronization image obtained immediately prior to the pre-synchronization image on which noise reduction processing is performed by the noise reduction processing unit 413 (hereinafter, a circular image). In the embodiment, the motion vector detection processing unit 412 detects the motion of the image as the motion vector by using the pre-synchronization image of a color component (luminance component) of the luminance component pixel selected by the luminance component pixel selecting unit 411 and the circular image. In other words, the motion vector detection processing unit 412 detects the motion of the image between the pre-synchronization image and the circular image at different imaging timings (captured in time series) as the motion vector.

The noise reduction processing unit 413 reduces a noise component of the pre-synchronization image (imaging signal) by weighted average processing between the images by using the pre-synchronization image and the circular image. The circular image is obtained by outputting the pre-synchronization image stored in the frame memory 414. The noise reduction processing unit 413 outputs the pre-synchronization image on which the noise reduction processing is performed to the frame memory 414.

The frame memory 414 stores image information of one frame forming one image (pre-synchronization image). Specifically, the frame memory 414 stores the information of the pre-synchronization image on which the noise reduction processing is performed by the noise reduction processing unit 413. In the frame memory 414, when the pre-synchronization image is newly generated by the noise reduction processing unit 413, the information is updated to that of the newly generated pre-synchronization image. The frame memory 414 may be formed of a semiconductor memory such as a video random access memory (VRAM) or a part of a storage area of the storage unit 43.

The demosaicing processing unit 415 determines an interpolating direction from correlation of color information (pixel values) of a plurality of pixels based on the imaging signal on which the noise reduction processing is performed by the noise reduction processing unit 413 and interpolates based on the color information of the pixels arranged in the determined interpolating direction, thereby generating a color image signal. The demosaicing processing unit 415 performs interpolation processing of the luminance component based on the luminance component pixel selected by the luminance component pixel selecting unit 411 and then performs the interpolation processing of the color component other than the luminance component, thereby generating the color image signal.

The display image generation processing unit 416 performs gradation conversion, magnification processing, emphasis processing of a blood vessel and vasculature of a living body and the like on the electric signal generated by the demosaicing processing unit 415. The display image generation processing unit 416 performs predetermined processing thereon and then outputs the same as the display image signal for display to the display unit 5.

The image processing unit 41 performs OB clamp processing, gain adjustment processing and the like in addition to the above-described demosaicing processing. In the OB clamp processing, processing to correct an offset amount of a black level is performed on the electric signal input from the endoscope 2 (A/D converter 205). In the gain adjustment processing, adjustment processing of a brightness level is performed on the image signal on which the demosaicing processing is performed.

The input unit 42 being an interface for inputting to the processor 4 by the user includes a power switch for turning on/off power, a mode switching button for switching between a shooting mode and various other modes, an illumination light switching button for switching the illumination light of the light source unit 3 (imaging mode) and the like.

The storage unit 43 records data including various programs for operating the endoscope device 1, various parameters required for the operation of the endoscope device 1 and the like. The storage unit 43 may also store a relation table between the information regarding the endoscope 2, for example, the specific information (ID) of the endoscope 2 and the information regarding the filter arrangement of the color filter 202a. The storage unit 43 is realized by using a semiconductor memory such as a flash memory and a dynamic random access memory (DRAM).

The control unit 44 formed of a CPU and the like performs driving control of each component including the endoscope 2 and the light source unit 3, input/output control of the information to/from each component and the like. The control unit 44 transmits the setting data (for example, the pixel to be read) for imaging control recorded in the storage unit 43, a timing signal regarding imaging timing and the like to the endoscope 2 through a predetermined signal line. The control unit 44 outputs color filter information (identification information) obtained through the imaging information storage unit 206 to the image processing unit 41 and outputs information regarding the arrangement of the switching filter 31c to the light source unit 3 based on the color filter information.

Next, the display unit 5 is described. The display unit 5 receives the display image signal generated by the processor 4 through a video cable to display the in-vivo image corresponding to the display image signal. The display unit 5 is formed of a liquid crystal, organic electro luminescence (EL) or the like.

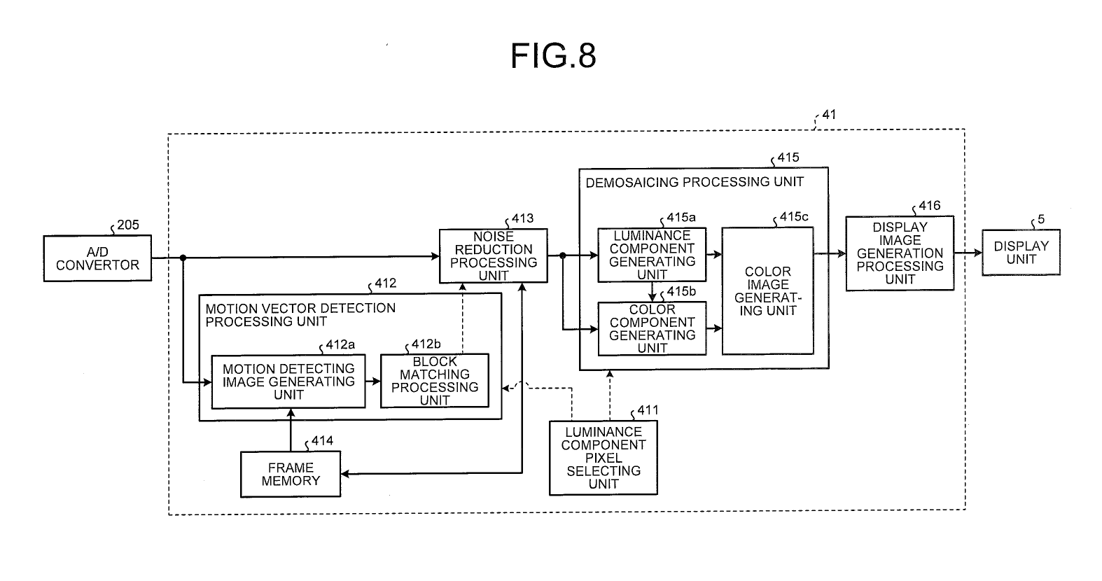

Subsequently, signal processing performed by each unit of the processor 4 of the endoscope device 1 is described with reference to FIG. 8. FIG. 8 is a block diagram illustrating a configuration of a substantial part of the processor of the endoscope device according to the embodiment.

The luminance component pixel selecting unit 411 determines the imaging mode out of the white light imaging mode and the narrow band imaging mode in which the input imaging signal is generated. Specifically, the luminance component pixel selecting unit 411 determines the imaging mode in which this is generated based on a control signal (for example, information regarding the illumination light and information indicating the imaging mode) from the control unit 44, for example.

When determining that the input imaging signal is generated in the white light imaging mode, the luminance component pixel selecting unit 411 selects the G pixel as the luminance component pixel to set and outputs the set setting information to the motion vector detection processing unit 412 and the demosaicing processing unit 415. Specifically, the luminance component pixel selecting unit 411 outputs positional information of the G pixel set as the luminance component pixel, for example, the information regarding the row and the column of the G pixel based on the identification information (information of the color filter 202a).

On the other hand, when determining that the input imaging signal is generated in the narrow band imaging mode, the luminance component pixel selecting unit 411 selects the B pixel as the luminance component pixel to set and outputs the set setting information to the motion vector detection processing unit 412 and the demosaicing processing unit 415.

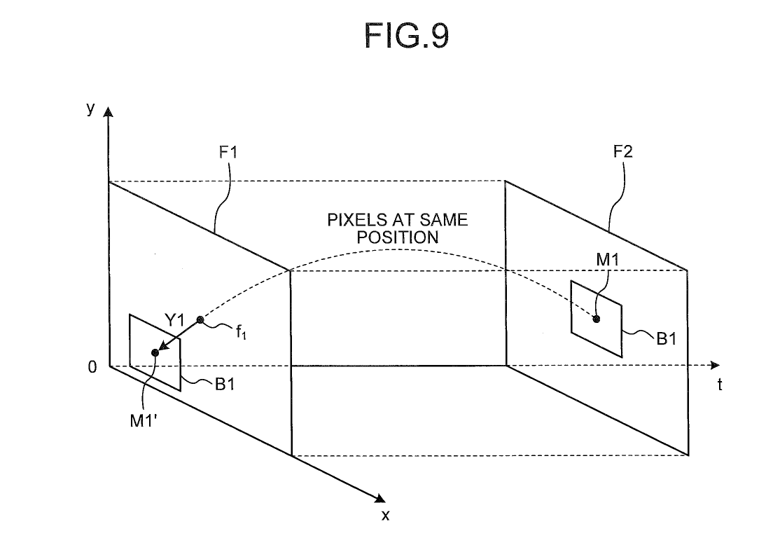

Next, processing performed by the motion vector detection processing unit 412 and the noise reduction processing unit 413 is described. FIG. 9 is a schematic view illustrating motion detection between images at different imaging timings (time t) performed by the motion vector detection processing unit of the endoscope device according to the embodiment of the present invention. As illustrated in FIG. 9, the motion vector detection processing unit 412 detects the motion of the image between a first motion detecting image F1 and a second motion detecting image F2 as the motion vector by using a well-known block matching method by using the first motion detecting image F1 based on the circular image and the second motion detecting image F2 based on the pre-synchronization image to be processed. The first and second motion detecting images F1 and F2 are the images based on the imaging signals of two consecutive frames in time series.

The motion vector detection processing unit 412 includes a motion detecting image generating unit 412a and a block matching processing unit 412b. The motion detecting image generating unit 412a performs the interpolation processing of the luminance component according to the luminance component pixel selected by the luminance component pixel selecting unit 411 to generate the motion detecting images (first and second motion detecting images F1 and F2) to which the pixel value or an interpolated pixel value (hereinafter, referred to as an interpolated value) of the luminance component is added according to each pixel. The interpolation processing is performed on each of the pre-synchronization image and the circular image. The method of the interpolation processing may be similar to that of a luminance component generating unit 415a to be described later.

The block matching processing unit 412b detects the motion vector for each pixel from the motion detecting images generated by the motion detecting image generating unit 412a by using the block matching method. Specifically, the block matching processing unit 412b detects a position in the first motion detecting image F1 where a pixel M1 of the second motion detecting image F2 moves, for example. The motion vector detection processing unit 412 sets a block B1 (small region) around the pixel M1 as a template, and scans the first motion detecting image F1 with the template of the block B1 around a pixel f₁ at the same position as the position of the pixel M1 of the second motion detecting image F2 in the first motion detecting image F1, to set a central pixel at a position where the sum of an absolute value of a difference between the templates is the smallest, as a pixel M1′. The motion vector detection processing unit 412 detects a motion amount Y1 from the pixel M1 (pixel f₁) to the pixel M1′ in the first motion detecting image F1 as the motion vector and performs this processing to all the pixels on which the image processing is to be performed. Hereinafter, coordinates of the pixel M1 are denoted by (x,y), and x and y components of the motion vector on the coordinates (x,y) are denoted by Vx(x,y) and Vy(x,y), respectively. When coordinates of the pixel M1′ in the first motion detecting image F1 are denoted by (x′,y′), x′ and y′ are defined by following formulae (1) and (2), respectively. The block matching processing unit 412b outputs information of the detected motion vector (including the positions of the pixels M1 and M1′) to the noise reduction processing unit 413.

x′=x+Vx(x,y) (1)

y′=y+Vy(x,y) (2)

The noise reduction processing unit 413 reduces the noise of the pre-synchronization image by the weighted average processing between the images, the pre-synchronization image and the circular image. Hereinafter, a signal after the noise reduction processing of a pixel of interest, such as the pixel M1 (coordinates (x,y)), is denoted by Inr(x,y). The noise reduction processing unit 413 refers to the motion vector information, determines whether a reference pixel corresponding to the pixel of interest is the pixel of the same color, and executes different processing for the cases of the same color and different colors. For example, the noise reduction processing unit 413 refers to information of the circular image stored in the frame memory 414 to obtain information (signal value and color information of transmission light) of the pixel M1′ (coordinates (x′,y′)) being the reference pixel corresponding to the pixel M1 and determines whether the pixel M1′ is the pixel of the same color as the pixel M1.

1). When Pixel of Interest and Reference Pixel Share Same Color

When the pixel of interest and the reference pixel share the same color (i.e., pixels for receiving the light of the same color component), the noise reduction processing unit 413 generates the signal Inr(x,y) by performing the weighted average processing using one pixel of each of the pre-synchronization image and the circular image by using following formula (3).

Inr(x,y)=coef×I(x,y)+(1−coef)×I′(x′,y′) (3)

where I(x,y) is a signal value of the pixel of interest of the pre-synchronization image, and

I′(x′,y′) is a signal value of the reference pixel of the circular image.

The signal value includes the pixel value or the interpolated value. A coefficient coef is an arbitrary real number satisfying 0<coef<1. The coefficient coef may be such that a predetermined value is set in advance or an arbitrary value is set by a user through the input unit 42.

2). When Pixel of Interest and Reference Pixel have Different Colors

When the pixel of interest and the reference pixel have different colors (i.e., pixels for receiving the light of different color components), the noise reduction processing unit 413 interpolates the signal value in the reference pixel of the circular image from a peripheral same color pixel. The noise reduction processing unit 413 generates the signal Inr(x,y) after the noise reduction processing by using following formula (4), for example.

$$Inr ( x , y ) = coef \times I ( x , y ) + ( 1 - coef ) \times \frac{{\sum }_{i = - K}^{K} {\sum }_{j = - K}^{K} w ( {x}^{′} + i , {y}^{′} + j ) \times {I}^{′} ( {x}^{′} + i , {y}^{′} + j )}{{\sum }_{i = - K}^{K} {\sum }_{j = - K}^{K} w ( {x}^{′} + i , {y}^{′} + j )} \qquad ( 4 )$$

If I(x,y) and I(x°+i,y′+j) are the signal values of the pixels of the same color, w(x′+i,y′+j)=1 is satisfied, and when I(x,y) and I(x′+i,y′+j) are the signal values of the pixels of the different colors, w(x′+i,y′+j)=0 is satisfied.

In formula (4), w( ) is a function for extracting the pixel of the same color which takes 1 when the peripheral pixel (x′+i,y′+j) is of the same color as the pixel of interest (x,y) and takes 0 when they are of different colors. K is a parameter which sets a size of a peripheral region to be referred to. The parameter K takes 1 at the time of the G pixel or the B pixel (K=1) and takes 2 at the time of the R pixel (K=2).

Subsequently, the interpolation processing by the demosaicing processing unit 415 is described. The demosaicing processing unit 415 generates the color image signal by performing the interpolation processing based on the signal (signal Inr(x,y)) obtained by performing the noise reduction processing by the noise reduction processing unit 413. The demosaicing processing unit 415 includes a luminance component generating unit 415a, a color component generating unit 415b, and a color image generating unit 415c. The demosaicing processing unit 415 determines the interpolating direction from the correlation of the color information (pixel values) of a plurality of pixels based on the luminance component pixel selected by the luminance component pixel selecting unit 411 and interpolates based on the color information of the pixels arranged in the determined interpolating direction, thereby generating the color image signal.

The luminance component generating unit 415a determines the interpolating direction by using the pixel value generated by the luminance component pixel selected by the luminance component pixel selecting unit 411 and interpolates the luminance component in the pixel other than the luminance component pixel based on the determined interpolating direction to generate the image signal forming one image in which each pixel has the pixel value or the interpolated value of the luminance component.

Specifically, the luminance component generating unit 415a determines an edge direction as the interpolating direction from a well-known luminance component (pixel value) and performs the interpolation processing in the interpolating direction on a non-luminance component pixel to be interpolated. When the B pixel is selected as the luminance component pixel, for example, the luminance component generating unit 415a calculates a signal value B(x,y) of the B component being the non-luminance component pixel in the coordinates (x,y) from following formulae (5) to (7) based on the determined edge direction.

When change in luminance in a horizontal direction is larger than that in a vertical direction, the luminance component generating unit 415a determines that the vertical direction is the edge direction and calculates the signal value B(x,y) by following formula (5).

$$B ( x , y ) = \frac{1}{2} \{ B ( x , y - 1 ) + B ( x , y + 1 ) \} \qquad ( 5 )$$

When determining the edge direction, an up-and-down direction of the arrangement of the pixels illustrated in FIG. 3 is made the vertical direction and a right-and-left direction thereof is made the horizontal direction. In the vertical direction, a downward direction is positive, and in the right-and-left direction, a rightward direction is positive.

When the change in luminance in the vertical direction is larger than that in the horizontal direction, the luminance component generating unit 415a determines that the horizontal direction is the edge direction and calculates the signal value B(x,y) by following formula (6).

$$B ( x , y ) = \frac{1}{2} \{ B ( x - 1 , y ) + B ( x + 1 , y ) \} \qquad ( 6 )$$

When difference between the change in luminance in the vertical direction and that in the horizontal direction is small (the change in luminance in both directions is flat), the luminance component generating unit 415a determines that the edge direction is neither the vertical direction nor the horizontal direction and calculates the signal value B(x,y) by following formula (7). In this case, the luminance component generating unit 415a calculates the signal value B(x,y) by using the signal values of the pixels located in the vertical direction and the horizontal direction.

$$B ( x , y ) = \frac{1}{4} \{ B ( x - 1 , y ) + B ( x + 1 , y ) + B ( x , y + 1 ) + B ( x , y - 1 ) \} \qquad ( 7 )$$

The luminance component generating unit 415a interpolates the signal value B(x,y) of the B component of the non-luminance component pixel by formulae (5) to (7) described above, thereby generating the image signal in which at least the pixel forming the image has the signal value (pixel value or interpolated value) of the signal of the luminance component.

On the other hand, when the G pixel is selected as the luminance component pixel, the luminance component generating unit 415a first interpolates a signal value G(x,y) of the G signal in the R pixel by following formulae (8) to (10) based on the determined edge direction. Thereafter, the luminance component generating unit 415a interpolates the signal value G(x,y) by the method similar to that of the signal value B(x,y) (formulae (5) to (7)).

When the change in luminance in an obliquely upward direction is larger than that in an obliquely downward direction, the luminance component generating unit 415a determines that the obliquely downward direction is the edge direction and calculates the signal value G(x,y) by following formula (8).

$$G ( x , y ) = \frac{1}{2} \{ G ( x - 1 , y - 1 ) + G ( x + 1 , y + 1 ) \} \qquad ( 8 )$$

When the change in luminance in the obliquely downward direction is larger than that in the obliquely upward direction, the luminance component generating unit 415a determines that the obliquely upward direction is the edge direction and calculates the signal value G(x,y) by following formula (9).

$$G ( x , y ) = \frac{1}{2} \{ G ( x + 1 , y - 1 ) + G ( x - 1 , y + 1 ) \} \qquad ( 9 )$$

When difference between the change in luminance in the obliquely downward direction and that in the obliquely upward direction is small (the change in luminance in both directions is flat), the luminance component generating unit 415a determines that the edge direction is neither the obliquely downward direction nor the obliquely upward direction and calculates the signal value G(x,y) by following formula (10).

$$G ( x , y ) = \frac{1}{4} \{ G ( x - 1 , y - 1 ) + G ( x + 1 , y - 1 ) + G ( x - 1 , y + 1 ) + G ( x + 1 , y + 1 ) \} \qquad ( 10 )$$

The method of interpolating the signal value G(x,y) of the G component (luminance component) of the R pixel in the edge direction (interpolating direction) (formulae (8) to (10)) is herein described, the method is not limited to this. Well-known bi-cubic interpolation may also be used as another method.

The color component generating unit 415b interpolates the color component of at least the pixel forming the image by using the signal values of the luminance component pixel and the color component pixel (non-luminance component pixel) to generate the image signal forming one image in which each pixel has the pixel value or the interpolated value of the color component. Specifically, the color component generating unit 415b calculates color difference signals (R-G and B-G) in positions of the non-luminance component pixels (B pixel and R pixel) by using the signal (G signal) of the luminance component (for example, the G component) interpolated by the luminance component generating unit 415a and performs well-known bi-cubic interpolation processing, for example, on each color difference signal. The color component generating unit 415b adds the G signal to the interpolated color difference signal and interpolates the R signal and the B signal for each pixel. In this manner, the color component generating unit 415b generates the image signal obtained by adding the signal value (pixel value or interpolated value) of the color component to at least the pixel forming the image by interpolating the signal of the color component. By this method, a high-frequency component of the luminance is superimposed on the color component and the image with high resolution may be obtained. The present invention is not limited to this; it is also possible to simply perform the bi-cubic interpolation processing on the color signal.

The color image generating unit 415c synchronizes the image signals of the luminance component and the color component generated by the luminance component generating unit 415a and the color component generating unit 415b, respectively, and generates the color image signal including the color image (post-synchronization image) to which the signal value of an RGB component or a GB component is added according to each pixel. The color image generating unit 415c assigns the signals of the luminance component and the color component to R, G, and B channels. Relationship between the channels and the signals in the imaging modes (WLI and NBI) will be hereinafter described. In the embodiment, the signal of the luminance component is assigned to the G channel.
WLI mode NBI mode

R channel: R signal G signal

G channel: G signal B signal

B channel: B signal B signal

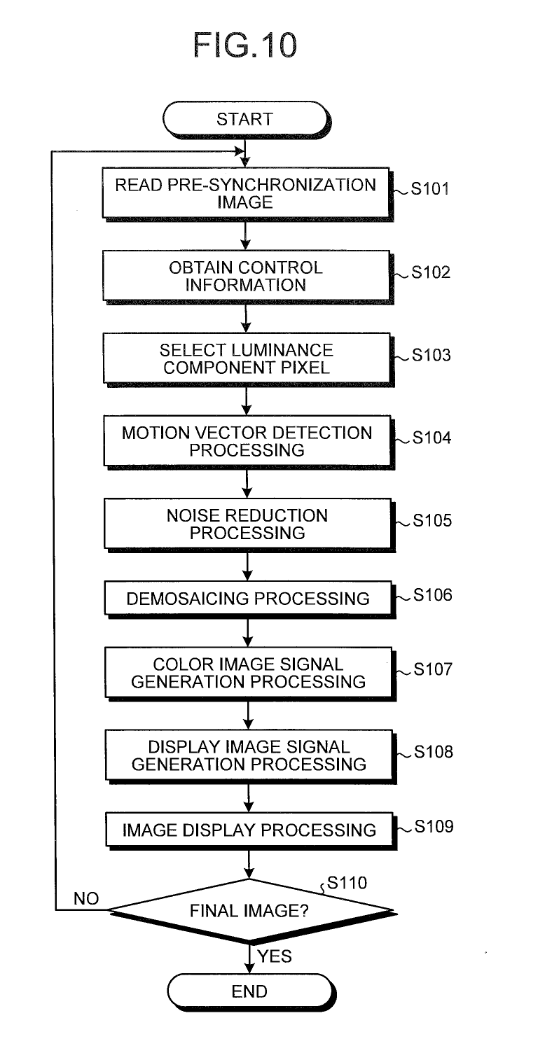

Subsequently, signal processing performed by the processor 4 having the above-described configuration is described with reference to the drawings. FIG. 10 is a flowchart illustrating the signal processing performed by the processor 4 of the endoscope device 1 according to the embodiment. When obtaining the electric signal from the endoscope 2, the control unit 44 reads the pre-synchronization image included in the electric signal (step S101). The electric signal from the endoscope 2 is the signal including the pre-synchronization image data generated by the image sensor 202 to be converted to a digital signal by the A/D converter 205.

After reading the pre-synchronization image, the control unit 44 refers to the identification information storage unit 261 to obtain the control information (for example, the information regarding the illumination light (imaging mode) and the arrangement information of the color filter 202a) and outputs the same to the luminance component pixel selecting unit 411 (step S102).

The luminance component pixel selecting unit 411 determines the imaging mode out of the obtained white light imaging (WLI) mode and narrow band imaging (NBI) mode in which the electric signal (read pre-synchronization image) is generated based on the control information and selects the luminance component pixel based on the determination (step S103). Specifically, when the luminance component pixel selecting unit 411 determines that the mode is the WLI mode, this selects the G pixel as the luminance component pixel, and when this determines that the mode is the NBI mode, this selects the B pixel as the luminance component pixel. The luminance component pixel selecting unit 411 outputs the control signal regarding the selected luminance component pixel to the motion vector detection processing unit 412 and the demosaicing processing unit 415.

When obtaining the control signal regarding the luminance component pixel, the motion vector detection processing unit 412 detects the motion vector based on the pre-synchronization image and the circular image of the luminance component (step S104). The motion vector detection processing unit 412 outputs the detected motion vector to the noise reduction processing unit 413.

The noise reduction processing unit 413 performs the noise reduction processing on the electric signal (pre-synchronization image read at step S101) by using the pre-synchronization image, the circular image, and the motion vector detected by the motion vector detection processing unit 412 (step S105). The electric signal (pre-synchronization image) after the noise reduction processing generated at this step S105 is output to the demosaicing processing unit 415 and stored (updated) in the frame memory 414 as the circular image.

When the electric signal after the noise reduction processing is input from the noise reduction processing unit 413, the demosaicing processing unit 415 performs the demosaicing processing based on the electric signal (step S106). In the demosaicing processing, the luminance component generating unit 415a determines the interpolating direction in the pixels to be interpolated (pixels other than the luminance component pixel) by using the pixel value generated by the pixel set as the luminance component pixel and interpolates the luminance component in the pixel other than the luminance component pixel based on the determined interpolating direction to generate the image signal forming one image in which each pixel has the pixel value or the interpolated value of the luminance component. Thereafter, the color component generating unit 415b generates the image signal forming one image having the pixel value or the interpolated value of the color component other than the luminance component for each color component based on the pixel value and the interpolated value of the luminance component and the pixel value of the pixel other than the luminance component pixel.

When the image signal for each color component is generated by the color component generating unit 415b, the color image generating unit 415c generates the color image signal forming the color image by using the image signal of each color component (step S107). The color image generating unit 415c generates the color image signal by using the image signals of the red component, the green component, and the blue component in the WLI mode and generates the color image signal by using the image signals of the green component and the blue component in the NBI mode.

After the color image signal is generated by the demosaicing processing unit 415, the display image generation processing unit 416 performs the gradation conversion, the magnification processing and the like on the color image signal to generate the display image signal for display (step S108). The display image generation processing unit 416 performs predetermined processing thereon and thereafter outputs the same as the display image signal to the display unit 5.

When the display image signal is generated by the display image generation processing unit 416, image display processing is performed according to the display image signal (step S109). The image according to the display image signal is displayed on the display unit 5 by the image display processing.

After the generation processing and the image display processing of the display image signal by the display image generation processing unit 416, the control unit 44 determines whether the image is a final image (step S110). The control unit 44 finishes the procedure when a series of processing is completed for all the images (step S110: Yes), or proceeds to step S101 to continuously perform the similar processing when the image not yet processed remains (step S110: No).

Although it is described that each unit forming the processor 4 is formed of hardware and each unit performs the processing in the embodiment, it is also possible to configure such that the CPU performs the processing of each unit and the CPU executes the program to realize the above-described signal processing by software. For example, it is possible that the CPU executes the above-described software to realize the signal processing on the image obtained in advance by the image sensor of a capsule endoscope and the like. A part of the processing performed by each unit may also be configured by the software. In this case, the CPU executes the signal processing according to the above-described flowchart.

According to the embodiment described above, in the color filter 202a provided on the image sensor 202, the filters are arranged by repeatedly arranging the filter arrangement of the filter unit U1 in which the numbers of the B filters and the G filters are larger than the number of the R filters as the basic pattern, so that the image with high resolution may be obtained both in the white light imaging mode and in the narrow band imaging mode.

According to the embodiment described above, it is possible to detect the motion between the images at a high degree of accuracy regardless of the imaging mode (NBI mode or WLI mode) by adoptively switching the motion vector detection processing by the motion vector detection processing unit 412 for the imaging mode. Specifically, the G pixel with which the blood vessel and the vasculature of the living body are clearly represented is selected as the luminance component pixel in the WLI mode and the motion vector between the images is detected by using the G pixel. On the other hand, in the NBI mode, the B pixel with which the blood vessel and the vasculature on a living body surface layer are clearly represented is selected as the luminance component pixel and the motion vector is detected by using the B pixel. By using the highly accurate motion vector obtained by such selection of the luminance component pixel, the noise reduction in which a residual image is inhibited becomes possible and the image with higher resolution may be obtained.

According to the embodiment described above, switching the demosaicing processing according to the imaging mode may further improve the resolution. Specifically, the G pixel is selected as the luminance component pixel in the WLI mode and the interpolation processing in the edge direction is performed on the G pixel. Furthermore, the G signal is added after the interpolation processing on the color difference signals (R-G and B-G) and the high-frequency component of the G signal is also superimposed on the color component. On the other hand, the B pixel is selected as the luminance component pixel in the NBI mode and the interpolation processing in the edge direction is performed on the B pixel. Furthermore, the B signal is added after the interpolation processing on the color difference signal (G-B) and the high-frequency component of the B signal is also superimposed on the color component. By the above-described configuration, the resolution may be improved as compared to the well-known bi-cubic interpolation. According to the configuration of the embodiment, the noise of the electric signal used for the demosaicing processing is reduced by the noise reduction processing unit 413 located on a preceding stage of the demosaicing processing unit 415, so that a degree of accuracy in determining the edge direction is advantageously improved.

First Modification

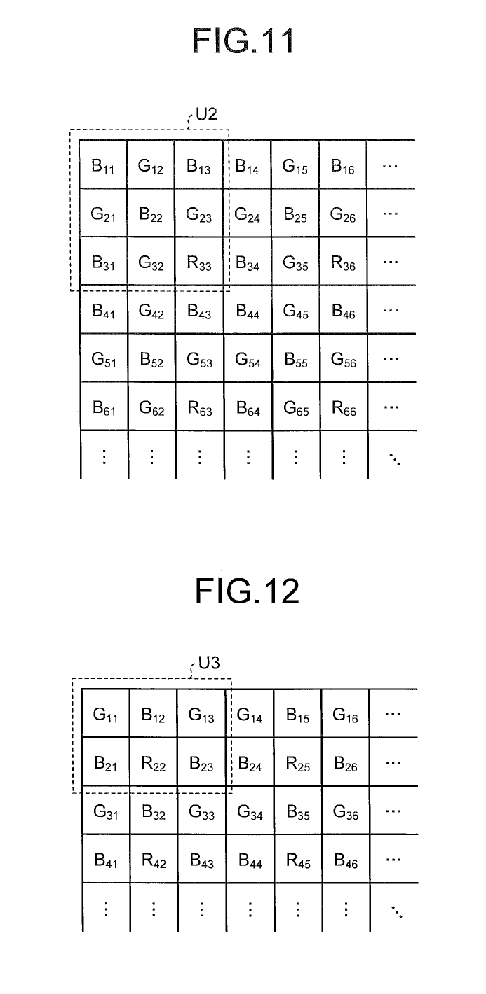

FIG. 11 is a schematic diagram illustrating a configuration of a color filter according to a first modification of the embodiment. The color filter according to the first modification is such that filter units U2, each of which is formed of nine filters arranged in a 3×3 matrix, are arranged in a two-dimensional manner. The filter unit U2 is formed of four B filters, four G filters, and one R filter. In the filter unit U2, the filters which transmit light of a wavelength band of the same color (same color filters) are arranged so as not to be adjacent to each other in a row direction and a column direction.

The filter unit U2 is such that the numbers of the B filters and the G filters are not smaller than one third of the total number of the filters (nine) forming the filter unit U2 and the number of the R filters is smaller than one third of the total number of the filters. In a color filter 202a (filter unit U2), a plurality of B filters forms a part of a checkerboard pattern.

Second Modification

FIG. 12 is a schematic diagram illustrating a configuration of a color filter according to a second modification of the embodiment. The color filter according to the second modification is such that filter units U3, each of which is formed of six filters arranged in a 2×3 matrix, are arranged in a two-dimensional manner. The filter unit U3 is formed of three B filters, two G filters, and one R filter. In the filter unit U3, the filters which transmit light of a wavelength band of the same color (same color filters) are arranged so as not to be adjacent to each other in a column direction and a row direction.

The filter unit U3 is such that the numbers of the B filters and the G filters are not smaller than one third of the total number of the filters (six) forming the filter unit U3 and the number of the R filters is smaller than one third of the total number of the filters.

Third Modification

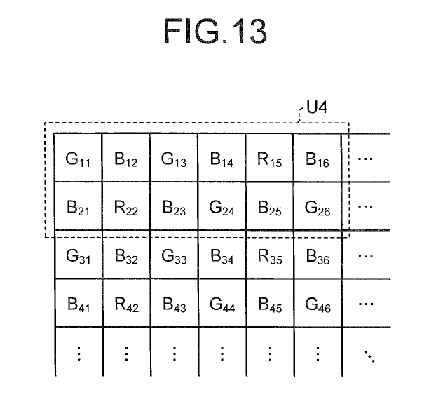

FIG. 13 is a schematic diagram illustrating a configuration of a color filter according to a third modification of the embodiment. The color filter according to the third modification is such that filter units U4, each of which is formed of 12 filters arranged in a 2×6 matrix, are arranged in a two-dimensional manner. The filter unit U4 is formed of six B filters, four G filters, and two R filters. In the filter unit U4, the filters which transmit light of a wavelength band of the same color (same color filters) are arranged so as not to be adjacent to each other in a row direction and a column direction, and a plurality of B filters is arranged in a zig-zag pattern.

The filter unit U4 is such that the numbers of the B filters and the G filters are not smaller than one third of the total number of the filters (12) forming the filter unit U4 and the number of the R filters is smaller than one third of the total number of the filters. In the color filter 202a (filter unit U4), a plurality of B filters is arranged in a checkerboard pattern.

The color filter 202a according to the above-described embodiment may be such that the number of the B filters which transmit the light of the wavelength band H_{B} and the number of the G filters which transmit the light of the wavelength band H_{G} are larger than the number of the R filters which transmit the light of the wavelength band H_{R} in the filter unit; in addition to the above-described arrangement, the arrangement satisfying the above-described condition may also be applied. Although the above-described filter unit has filters that are arranged in a 4×4 matrix, a 3×3 matrix, a 2×3 matrix, or a 2×6 matrix, the numbers of the rows and columns are not limited thereto.

Although the color filter 202a including a plurality of filters, each of which transmits the light of a predetermined wavelength band, is provided on the light receiving surface of the image sensor 202 in the above-described embodiments, each filter may also be individually provided on each pixel of the image sensor 202.

Although the endoscope device 1 according to the above-described embodiment is described to switch the illumination light emitted from the illuminating unit 31 between the white illumination light and the narrow band illumination light by putting/removing the switching filter 31c for the white illumination light emitted from one light source 31a, it is also possible to switch between two light sources which emit the white illumination light and the narrow band illumination light to emit any one of the white illumination light and the narrow band illumination light. When the two light sources are switched to emit any one of the white illumination light and the narrow band illumination light, it is also possible to apply to the capsule endoscope provided with the light source unit, the color filter, and the image sensor, for example, introduced into the subject.

Although it is described that the A/D converter 205 is provided on the distal end portion 24 of the endoscope device 1 according to the above-described embodiment, this may also be provided on the processor 4. The configuration regarding the image processing may also be provided on the endoscope 2, a connector which connects the endoscope 2 to the processor 4, and the operating unit 22. Although it is described that the endoscope 2 connected to the processor 4 is identified by using the identification information and the like stored in the identification information storage unit 261 in the above-described endoscope device 1, it is also possible to provide an identifying unit on a connecting portion (connector) between the processor 4 and the endoscope 2. For example, a pin for identification (identifying unit) is provided on the endoscope 2 to identify the endoscope 2 connected to the processor 4.

Although it is described that the motion vector is detected after the synchronization regarding the luminance component by the motion detecting image generating unit 412a in the above-described embodiment, the present invention is not limited thereto. As another method, it may also be configured such that the motion vector is detected from the luminance signal (pixel value) before the synchronization. In this case, when matching is performed between the pixels of the same color, the pixel value cannot be obtained from the pixel other than the luminance component pixel (non-luminance component pixel), so that a matching interval is limited; however, an operational cost required for block matching may be reduced. Herein, the motion vector is detected only for the luminance component pixel, so that it is required to interpolate the motion vector in the non-luminance component pixel. For the interpolation processing at that time, the well-known bi-cubic interpolation may be used.

Although it is configured to perform the noise reduction processing on the pre-synchronization image before the demosaicing processing by the demosaicing processing unit 415 in the above-described embodiment, it is also possible that the noise reduction processing unit 413 performs the noise reduction processing on the color image output from the demosaicing processing unit 415. In this case, since all the reference pixels are the same color pixels, arithmetic processing of formula (4) is not required and it is possible to reduce the operational cost required for the noise reduction processing.

According to some embodiments, it is possible to obtain an image with high resolution both in a white light imaging mode and in a narrow band imaging mode.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope device comprising:
a light source unit configured to emit white illumination light including rays of light of red, green, and blue wavelength bands, or to emit narrow band illumination light having narrow band light included in each of the blue and green wavelength bands;
an image sensor that has a plurality of pixels arranged in a matrix pattern and is configured to perform photoelectric conversion on light received by each of the plurality of pixels to generate an electric signal;
a color filter having a plurality of filter units arranged on a light receiving surface of the image sensor, each of the filter units being formed of blue filters for transmitting the light of the blue wavelength band, green filters for transmitting the light of the green wavelength band, and red filters for transmitting the light of the red wavelength band, the number of the blue filters and the number of the green filters being larger than the number of the red filters;
a luminance component pixel selecting unit configured to select a luminance component pixel for receiving light of a luminance component, from the plurality of pixels according to types of illumination light emitted by the light source unit; and
a demosaicing processing unit configured to generate a color image signal having a plurality of color components based on the luminance component pixel selected by the luminance component pixel selecting unit.

2. The endoscope device according to claim 1, wherein
each of the filter units is configured such that
the number of the blue filters and the number of the green filters are not smaller than one third of a total number of filters constituting each of the filter units, and
the number of the red filters is smaller than one third of the total number of the filters.

3. The endoscope device according to claim 1, wherein
the blue filters forms at least a part of a checkerboard pattern.

4. The endoscope device according to claim 1, wherein
the luminance component pixel selecting unit is configured to:
select, as the luminance component pixel, a pixel for receiving the light through the green filter when the light source unit emits the white illumination light; and
select, as the luminance component pixel, a pixel for receiving the light through the blue filter when the light source unit emits the narrow band illumination light.

5. The endoscope device according to claim 1, wherein
the demosaicing processing unit comprises:
a luminance component generating unit configured to interpolate a luminance component of a pixel other than the luminance component pixel based on a pixel value of the luminance component pixel selected by the luminance component pixel selecting unit, thereby to generate an image signal of the luminance component; and
a color component generating unit configured to interpolate a color component other than the luminance component based on the luminance component generated by the luminance component generating unit, thereby to generate an image signal of the color component.

6. An endoscope device comprising:
a light source unit configured to emit white illumination light including rays of light of red, green, and blue wavelength bands, or to emit narrow band illumination light having narrow band light included in each of the blue and green wavelength bands;
an image sensor that has a plurality of pixels arranged in a matrix pattern and is configured to perform photoelectric conversion on light received by each of the plurality of pixels to generate an electric signal;
a color filter having a plurality of filter units arranged on a light receiving surface of the image sensor, each of the filter units being formed of blue filters for transmitting the light of the blue wavelength band, green filters for transmitting the light of the green wavelength band, and red filters for transmitting the light of the red wavelength band, the number of the blue filters and the number of the green filters being larger than the number of the red filters;
a luminance component pixel selecting unit configured to select a luminance component pixel for receiving light of a luminance component, from the plurality of pixels according to types of illumination light emitted by the light source unit; and
a motion detection processing unit configured to detect motion of a captured image generated based on the electric signal generated by the pixels in time series, the electric signal being of the luminance component selected by the luminance component pixel selecting unit.

7. The endoscope device according to claim 6, wherein
the luminance component pixel selecting unit is configured to:
select, as the luminance component pixel, a pixel for receiving the light through the green filter when the light source unit emits the white illumination light; and
select, as the luminance component pixel, a pixel for receiving the light through the blue filter when the light source unit emits the narrow band illumination light.

8. The endoscope device according to claim 6, further comprising a noise reduction processing unit configured to reduce a noise component included in the captured image based on the motion detected by the motion detection processing unit.
